SEQUENCE LISTING (1 sequence)

SEQ ID NO: 1 - protein, 13 aa, Artificial Sequence
  PADRE SEQUENCE
AKXVAAWTLKAAA

(19) United States
(12) Patent Application Publication
(10) Pub. No.: US 2013/0052204 A1
(43) Pub. Date: Feb. 28, 2013

(54) (POLY)-GLYCEROLPHOSPHATE-BASED ANTI-GRAM POSITIVE BACTERIAL VACCINE

(71) Applicants: Clifford M. Snapper, Potomac, MD (US); Andrew Lees, Silver Spring, MD (US); James J. Mond, Silver Spring, MD (US); David Schwartz, Enanitos, CA (US)

(72) Inventors: Clifford M. Snapper, Potomac, MD (US); Andrew Lees, Silver Spring, MD (US); James J. Mond, Silver Spring, MD (US); David Schwartz, Enanitos, CA (US)

(21) Appl. No.: 13/508,860

(22) PCT Filed: Nov. 15, 2010
(86) PCT No.: PCT/US10/56742

§ 371 Date: Jul. 24, 2012

Related U.S. Application Data
(60) Provisional application No. 61/261,572, filed on Nov. 16, 2009.

(51) Int. Cl. A61K 39/385; A61P 31/04; C07F 9/09; C07K 17/08; A61K 39/40

(52) U.S. Cl. 424/165.1; 530/327; 530/406; 424/197.11; 424/164.1; 558/156

(57) ABSTRACT

Provided are an immunogenic composition comprising polyglycerol phosphate (PGP) and methods for using the composition for treating or preventing staphylococcal infections. The PGP may be conjugated to a T-cell dependent antigen. Also provided are methods for synthesizing PGP and methods for conjugating PGP to a T-cell dependent antigen.

### STATEMENT OF GOVERNMENT INTEREST

This invention was made in part with support from the U.S. Government. Accordingly, the Government may have certain rights in this invention.

### FIELD OF THE INVENTION

The present invention relates to the field of immunogenic compositions and vaccines, their manufacture, and their use for the treatment and/or prevention of Gram-positive bacterial infections. More particularly, the invention relates to vaccine compositions comprising PGP antigens. Methods for preparing and using such compositions are also provided.

### BACKGROUND OF THE INVENTION

Most pathogenic bacteria in humans are Gram-positive organisms. Examples of Gram-positive bacteria include staphylococci, streptococci, corynebacterium, listeria, bacillus, and clostridium. Staphylococci normally inhabit and colonize the skin and mucus membranes of humans and other animals. If the skin or mucus membrane harboring the bacteria becomes damaged during surgery or other trauma, the staphylococci may gain access to internal tissues causing infection. If the staphylococci proliferate locally or enter the lymphatic or blood systems, serious infectious complications may result. Staphylococci are the leading cause of bacteremia, surgical wound infections, and infection of prosthetic materials in the United States, and the second leading cause of other hospital-acquired (nosocomial) infections. Complications associated with staphylococcal infections include septic shock, endocarditis, arthritis, osteomyelitis, pneumonia, and abscesses in various organs.

Staphylococci are classified as either coagulase-positive (CoPS) or coagulase-negative (CoNS). Staphylococcus aureus is the most common coagulase-positive form of staphylococci. S. aureus is the leading cause of surgical site infections (SSI) in community hospitals, causing 300,000 to 500,000 SSIs each year in the United States. Overall, S. aureus-induced SSIs account for $1 billion to $10 billion in health costs annually. S. aureus strains that are resistant to the antibiotic methicillin (MRSA strains) are responsible for 40% to 60% of nosocomial staphylococcal infections in the United States. MRSA strains increased from 9% to 49% between 1992 and 2002. From 2001 to 2003 there were 11.6 million ambulatory care visits for skin and soft tissue infections in the United States, many or most of which were thought to be due to MSRA strains. This emergence of community-acquired MRSA infections has heightened concern about the microbe and has lent new urgency to efforts to control the spread of staphylococci.

Coagulase-negative staphylococci are the most common cause of nosocomial bacteremia (30-40% of all cases). Approximately 250,000 cases of CoNS bacteremia occurs annually in the United States with appreciable morbidity, mortality ranging from 1-2% to 25%, an average additional cost per episode of $25,000, and prolongation of hospital stay by at least seven days. Staphylococcus epidermidis is the most commonly isolated coagulase-negative form of staphylococci, and is a major cause of clinically significant infections, largely due to its ability to grow on virtually all biomaterials used for indwelling medical devices. Once established, these infections tend to be unresponsive to antimicrobials, and often necessitate removal of the infected device.

Staphylococcus infections are typically treated with antibiotics. However, the percentage of staphylococcal strains exhibiting wide-spectrum resistance to antibiotics has become increasingly prevalent, decreasing the effectiveness of antimicrobial therapies. Although new antimicrobial agents are under investigation, it is expected that the bacteria ultimately will devise resistance mechanisms to circumvent these new antibiotics. Thus, there is a pressing need for non-antimicrobial approaches to preventing and/or treating staphylococci infections.

Human immunity to extracellular Gram-positive bacterial pathogens is primarily mediated by opsonic killing via antibodies specific for surface polysaccharides. (Skurnik D. et al., J. Clin. Invest., 120(9):3220-33 (2010).) S. aureus expresses two such antigens: capsular polysaccharide (CP) and poly-N-acetyl glucosamine (PNAG). Capsular polysaccharides represent the best established targets for vaccine-induced immunity to bacterial cells. (Skurnik D. et al., J. Clin. Invest., 120(9):3220-33 (2010).) However, due to a lack of knowledge as to what constitutes protective human immunity to staphylococcal infections, it has been difficult to use a rational approach to develop a suitable vaccine. For example, S. aureus produces various molecules with seemingly redundant functions, such that if one is eliminated (or targeted by a vaccine), other bacterial products may compensate for that loss of function. In addition, staphylococci have developed a number of diverse strategies to avoid human innate immunity. (Schaffer A. C. et al., Infect. Dis. Clin., 23:153-71 (2009).)

The approach of using antibodies against staphylococcal antigens in passive immunotherapy has been investigated with some preliminary success. For example, Phase 2 and 3 trials using antibodies to S. aureus capsular polysaccharides serotype 5 (CP5) and serotype 8 (CP8) (i.e., Altastaph), clumping factor A (ClfA (i.e., Aurexis), ATP-binding cassette (ABC) (i.e., Aurograb), and lipoteichoic acid (LTA) (i.e., Pagibaximab) have been completed. (Schaffer A. C. et al., Infect. Dis. Clin., 23:153-71 (2009).) However, a Phase 3 trial using a pooled human immunoglobulin preparation from donors with high antibody titers against staphylococcal adhesins that bind fibrinogen and fibrin (S. aureus ClfA and S. epidermis SdrG) (i.e., Veronate) has failed. This failure was particularly disappointing, because the antibody cocktail, although selected for antibodies to ClfA and SdrG, likely contained antibodies to many other staphylococcal antigens and, therefore, represents a failed attempt at multicomponent passive immunotherapy. (Schaffer A. C. et al., Infect. Dis. Clin., 23:153-71 (2009).)

To date, only two active immunization approaches involving administration of staphylococcal antigens have been tested in Phase 2 and 3 trials. One approach, based on the S. aureus capsular polysaccharides CP5 and CP8 in the form of a conjugate vaccine (i.e., StaphVax) failed at the Phase 3 stage. The vaccine failed to confer significant protection when administered to hemodialysis patients. The failure of this trial has led investigators to question whether it is possible to develop an effective staphylococci vaccine. (Schaffer A. C. et al., Infect. Dis. Clin., 23:153-71 (2009).) Indeed, recovery from an S. aureus infection does not appear to confer immunity against subsequent infections, suggesting immunity to staphylococci infection may not occur. Nevertheless, another vaccine based on the S. aureus cell wall-anchored protein, IsdB (i.e., V710), which is expressed only under conditions of limiting iron, recently entered Phase 2/3 testing. Currently, however, there exists no anti-Staphylococcal vaccine in clinical use, and no way to predict which bacterial components will confer protection if included in a vaccine.

Lipoteichoic acid (LTA) is a major component of all Gram-positive bacterial cell membranes that projects into the bacterial cell wall, and appears to be critical for bacterial function. (Deininger S. et al., J. Immunol., 170:4134-38 (2003).) There is also increasing evidence that LTA is immunostimulatory. For example, LTA has been shown to elicit a protective anti-bacterial effect following immunization. (Yokoyama Y. et al., Int J Pediatr Otorhinolaryngol, 63:235-241 (2002); Caldwell J. et al., J Med Microbiol., 15:339-350 (1982).) However, in U.S. Publication No. 2005/0169941, one of the inventors showed that natural purified LTA and deacylated natural purified LTA (deAcLTA) were only poorly immunogenic in mice. To improve immunogenicity, deAcLTA was linked to maleimide derivatized-tetanus toxoid (TT). The deAcLTA-TT conjugate vaccine induced high levels of anti-LTA IgG antibodies. In addition, the response was boostable, indicating conversion of the deAcLTA from a T-cell independent to a T-cell-dependent antigen. The antibodies induced by deAcLTA-TT cross-reacted with intact LTA, and the sera were highly protective in an opsophagocytic assay against S. epidermidis bacteria. Mice immunized with deAcLTA-TT were also resistant to intravenous (i.v.) infection with live S. aureus as manifested by marked diminution of bacteria in spleen and kidney.

The inventors, in conjunction with others, have also shown that a chimeric mouse/human monoclonal antibody against S. aureus LTA (Pagibaximab) was opsonic (i.e., enhanced phagocytosis) in vitro for S. epidermidis and S. aureus, and was protective in vivo against S. aureus. The Pagibaximab antibody was developed by immunizing mice with whole staphylococci and selecting the resulting monoclonocal antibodies from the fusion of spleen cells based on their ability to induce opsonization of staphylococci. Pagibaximab had the highest binding activity to the bacteria and induced high levels of opsonization. (Weisman L. E. et al., Int Immunopharmacol., 9:639-644 (2009); and Weisman L. E. et al., Antimicrob Agents Chemother., 53:2879-2886 (2009).) However, administration of LTA elicits an inflammatory reaction, making it an undesirable candidate for use in active vaccines. (See, e.g., Deininger S. et al., Clin. Vaccine Immunol., 14(12):1629-33 (2007); Morath S. et al., J. Endotoxin Res., 11(6):348-56 (2005); Deininger S. et al., J. Immunol., 170(8):4134-38 (2003); and Morath S. et al., J. Exp. Med., 195(12):1635-40 (2002).)

Accordingly, it is a primary object of the invention to address the needs in the field by providing immunogenic compositions and vaccines for the treatment and/or prevention of Gram-positive bacterial infection. Methods for preparing and using such compositions are also provided.

### SUMMARY OF THE INVENTION

The invention provides an immunogenic composition comprising poly-glycerolphosphate (PGP), a moiety that was not previously known to be a protective epitope for staphylococcal infection.

In one aspect, the PGP is covalently linked to a T-cell dependent antigen. In yet another aspect, the T-cell dependent antigen is tetanus toxoid (TT), diptheria toxoid (DT), genetically detoxified diphtheria toxin, pertussis toxoid (PT), recombinant exoprotein A (rEPA), outer membrane protein complex (OMPC), or a Pan DR helper T cell epitope (PADRE) peptide. In yet another aspect, the PADRE peptide comprises the sequence AKXVAAWTLKAAA, wherein X is cyclohexylalanine. In yet another aspect, the genetically detoxified diphtheria toxin is CRM 197.

In another aspect, the molar ratio of PGP to the T-cell dependent antigen is about 5:1 to 50:1. In yet another aspect, the molar ratio is 10:1.

In another aspect, the PGP is directly linked to the T-cell dependent antigen. In yet another aspect, the PGP is linked to the T-cell dependent antigen through a linker.

In another aspect, the PGP is covalently linked to the T-cell dependent antigen using a thiol group, a thiol-ether group, an acyl-hydrazone group, a hydrazide group, a hydrazine group, a hydrazone, especially a bis-arylhydrazone group, or an oxime group. In yet another aspect, the thiol nucleophile group is incorporated using, for example succinimidyl 6-[3-(2-pyridyldithio)-propionamido]hexanoate (SPDP), or N-succinimidyl-S-acetylthioacetate (SATA). In yet another aspect, the hydrazide nucleophile group is added using E-maleimidocaprioc acid hydrazide-HCl (EMCH), or hydrazine or adipic dihydrazide (ADH) and 1-ethyl-3-dimethylaminopropyl)carbadiimide hydrochloride (EDC) and the arylhydrazine group is added using succinimidyl hydrazinonicotinate acetone hydrazone (S-HyNic, Solulink Biosciences, San Diego, Calif.).

In another aspect, the PGP is synthetic. In yet another aspect, the PGP is produced by preparing a substituted phosphoramidite monomer and elongating it stepwise using standard solid phase nucleic acid technology.

In another aspect, the PGP comprises about 5-20 glycerol phosphate monomers. In yet another aspect, the PGP comprises about 10-12 glycerol phosphate monomers. In yet another aspect, the PGP comprises about 10 glycerol phosphate monomers.

The invention also provides a method for treating an infection by a bacteria expressing a PGP moiety, a method for vaccinating a subject against a bacteria expressing a PGP moiety, and a method for generating protective antibodies against a bacteria expressing a PGP moiety, said methods comprising administering an effective amount of an immunogenic composition of the invention.

In one aspect, the bacteria is staphylococci. In another aspect, the bacteria is Staphylococcus aureus or Staphylococcus epidermidis.

In one aspect, the immunogenic composition is administered parenterally. In another aspect, the immunogenic composition is administered with another active agent. In yet another aspect, the other active agent is an antibiotic, a bacterial antigen, or an anti-bacterial antibody.

The invention also provides a novel method for synthesizing poly-glycerolphosphate (PGP) by preparing a protected and activated phosphoramidite monomer and elongating it stepwise. In one aspect, the elongation comprises standard solid phase oligonucleotide synthetic technology. In another aspect the elongation is performed on a DNA synthesizer. In yet another aspect, a linking group is incorporated on the PGP during the elongation. In yet another aspect, the linking group is incorporated by a solid support during the elongation. In yet another aspect, the monomer contains a linking group or a precursor to a linking group. In yet another aspect, the linking group is an amino group.

In one aspect, the monomer is a glycerol molecule comprising (a) an acid labile protecting group on one terminal hydroxyl group; (b) a base labile group on the 2-OH; and/or (c) an activated phosphorus group on the other terminal hydroxyl. In another aspect, the monomer is prepared by (a) protecting a glycerol molecule with an acid labile protecting group on one terminal hydroxyl group; (b) protecting a glycerol with a base labile group on the 2-OH; and/or (c) protecting a glycerol with an activated phosphorus group on the other terminal hydroxyl.

In yet another aspect the glycerol is chirally pure. In another aspect, the activated phosphorus group contains a linking group or a precursor to a linking group. In yet another aspect, the linking group is an amino group. In yet another aspect, the base labile group of (b) is stable to acid deprotection conditions. In yet another aspect, the elongation comprises standard solid phase oligonucleotide synthetic technology using a solid phase support, wherein the base labile group of (b) is removed during the cleavage of PGP from the solid phase support. In yet another aspect, the glycerol molecule is first protected with the acid labile protecting group and then protected with the base labile group.

In yet another aspect, the monomer is prepared by (a) preparing a levulinate ester from an isopropylidene glycerol molecule; (b) removing the isopropylidene protecting group; (c) protecting the free terminal alcohol with an acid labile group; (d) protecting the 2-OH group with a base labile group; (e) deprotecting the levulinate ester to provide a free terminal hydroxyl; and (f) phosphitylating the free terminal alcohol. In one aspect, the isopropylidene glycerol molecule is chirally pure. In another aspect, the levulinate ester is removed by hydrazine.

The invention also provides a synthetic poly-glycerolphosphate (PGP) molecule produced by the method of the invention. The invention also provides a synthetic poly-glycerolphosphate (PGP) molecule comprising a linker. In one aspect, the synthetic PGP comprises a linker group. In another aspect, the linker group contains a thiol, amine, aminooxy, aldehyde, hydrazide, hydrazine, maleimide, carboxyl, or haloacyl.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed. The disclosures of any documents cited therein are hereby incorporated by reference.

The accompanying drawings, which are incorporated in and constitute a part of this specification, illustrate exemplary embodiments of the invention and together with the description, serve to explain the principles of the invention.

### BRIEF DESCRIPTION OF THE DRAWINGS

FIG. 1 shows the structure of Staphylococcus aureus lipoteichoic acid (LTA) and its poly(glycerolphosphate) (PGP) component.

FIG. 2 is the synthetic scheme employed to prepare PGP using a protected glycerol-phosphate phosphoramidite.

FIG. 3 shows the immunogenicity of deAcLTA in combination with TT (deAcLTA+TT) and TT-conjugated deAcLTA (deAcLTA-TT). Groups of 20 BALB/c mice were immunized on days 0, 14 and 28 with 5 ug of LTA mixed with TT, or conjugated to TT, and with Ribi adjuvant. Individual sera (day 28) were assayed for anti-LTA IgG by ELISA.

FIG. 4 shows BALB/c mice immunized with PGP-TT are specifically protected against infection with S. aureus. BALB/c mice (5 per group) were immunized with PGP-TT or PPS14-TT (1 μg/mouse) adsorbed on 13 mg of alum mixed with 25 μg of a stimulatory CpG-containing oligodeoxynucleotide (CpG-ODN) and similarly boosted on day 14. On day 36 mice were infected i.p. with 1.7×10⁷ CFU live S. aureus. Blood was obtained from the tail vein on days 1, 2, and 3 for determination of S. aureus colony counts.

### DETAILED DESCRIPTION OF THE INVENTION

The invention relates to immunogenic compositions and vaccines comprising PGP antigens, a moiety that was not previously known to be a protective epitope for staphylococcal infection. Methods for preparing and using such compositions for the treatment and/or prevention of infection by Gram-positive bacteria that express a PGP moiety are also provided.

### The PGP Antigen

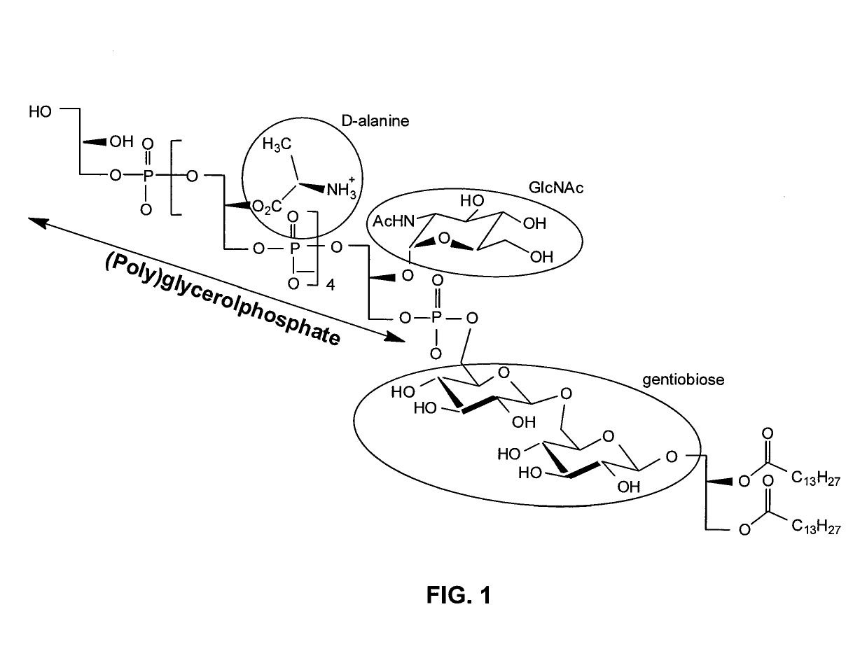

The structure of lipoteichoic acid (LTA) varies among bacteria but typically contains a core chain of poly-glycerolphosphate (PGP) (FIG. 1) or poly-ribitolphosphate (PRP) and a glycolipid tail. The PGP chains have pendant sugars and D-alanine esters on the glycerol. Staphylococci containing PGP include S. aureus and S. epidermidis. Staphylococci lacking PGP include S. citreus.

The inventors have now determined that the chimeric mouse/human monoclonal antibody against S. aureus LTA, Pagibaximab binds equally well to LTA from S. aureus and synthetic PGP (see Example 2), suggesting that these antibodies are specific for PGP. These new results suggest that PGP could serve as a target antigen for protection and/or treatment of staphylococci infections.

Accordingly, in one aspect the invention relates to immunogenic compositions comprising PGP. In another aspect, the PGP is synthetic. In yet another aspect, the PGP is covalently linked (i.e., conjugated) to an immunogenic protein capable of recruiting CD4+ helper T cells.

### Multivalent Antigens

Multivalent antigens, such as PGP, have been shown to be more potent stimulators of B cell receptor signaling and B cell activation than paucivalent antigens. For example, it was found that to be immunogenic, the type 2 T cell independent (TI-2) Ag DNP-polyacrylamide should exceed a threshold molecular mass of 100,000 Da and a threshold hapten valence of 20. (Dintzis R. Z. et al., J. Immunol., 131:2196-203 (1983).) The relationship between immunogenicity of a particular multivalent antigen and its molecular weight (i.e., number of repeating units) exhibits a bell curve for induction of antigen-specific immunoglobulin. (Dintzis R. Z. et al., J. Immunol., 143:1239-44 (1989).) Historically, multivalent antigens containing about 1-20 repeating units have been found to be highly immunogenic and serve as effective vaccine antigens.

Accordingly, in one aspect of the invention, the PGP comprises about 1-20 glycerol phosphate monomers. In another aspect, the PGP comprises about 5-10 glycerol phosphate monomers. In yet another aspect, the PGP comprises about 1, 2, 3, 4, 5, 6, 7, 8, 9, 10, 11, 12, 13, 14, 15, 16, 17, 18, 19, or 20 glycerol phosphate monomers.

### PGP Synthesis

Preparation of appropriately protected glycerol amidites suitable for elongation by solid phase nucleic acid synthesis is difficult, since the activated monomer must incorporate an acid labile alcohol protecting group on one terminus, a base labile group on the 2-OH that is stable to acid deprotection conditions, and an active phosphoramidite group for elongation. As used herein, the terms “active” and “activated” mean that at least one moiety on a chemical entity has been rendered capable of interacting with another molecule, for example, through one or more covalent bonds.

In one aspect, the acid labile group is dimethoxytrityl. The acid labile group may be removed using trichloroacetic acid or dichloroacetic acid. In another aspect, the base labile group is a levulinate group. The base labile group may be removed, for example, by hydrazine. In yet another aspect, the active phosphoramidite group may be a 3-(((diisopropylamino)phosphino)oxy)propanenitrile group.

A chirally pure glycerol monomer possessing the (S)-(+) configuration increases the difficulty of finding a synthetic route to a commercially available appropriate monomer. The difficulty of finding a suitable route can be understood by one skilled in the art as (a) three hydroxyl protecting groups that can be removed without affecting any other protecting must be found, and (b) the choice of suitable protecting groups is limited, as the hydroxyl groups are alpha to each other, which allows ready migration of a protecting group under certain conditions.

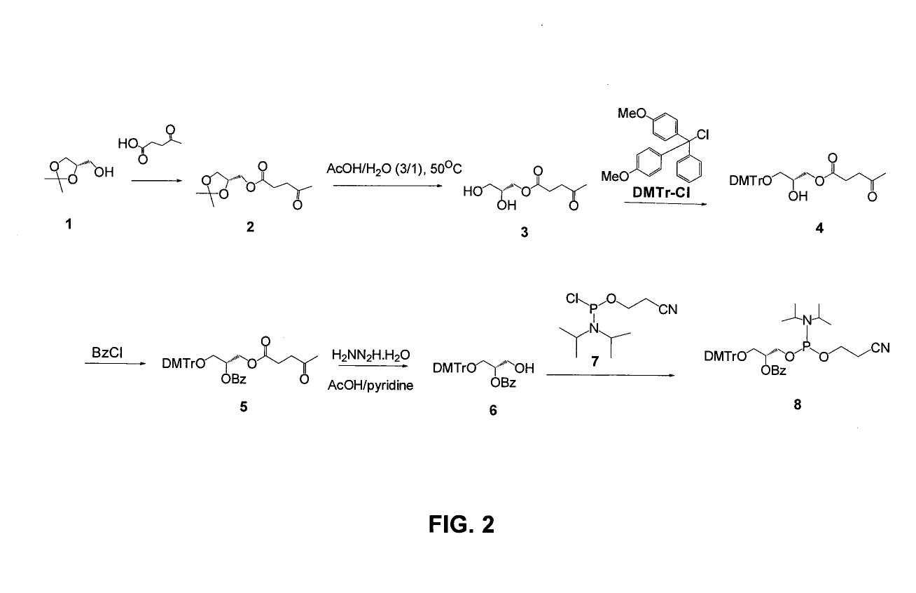

With this in mind, the inventors evaluated multiple routes of synthesizing PGP, and have developed a reliable method for synthesizing PGP in high quantities, as provided in the general outline in FIG. 2 and Example 1. Accordingly, the phosphoramidite may be synthesized from (S)-(+)-1,2-isopropylideneglycerol in six steps comprising: (a) protection of the 3-OH group as its levulinate ester, (b) acid deprotection of the isopropylidene protecting group, (c) incorporation of a dimethoxytrityl (DMTr) group on the 1-OH, (d) protection of the 2-OH as a benzoate, (e) removal of the 3-O-levulinate group, and (f) phosphitylation of the 3-OH. While the specific protecting groups mentioned above may lead to high yields, other acid and base labile groups may be used so long as they are able to be removed without affecting any other protecting and without suffering from migration problems. Additionally, while chirally pure isopropylidine glycerol is an option, the racemate may also be used.

In another aspect of the invention, elongation may occur, for example, through the use of standard solid phase nucleic acid synthetic protocols on a DNA synthesizer. In another aspect, a phosphoramidite, for example, a 1-O-dimethoxytrityl group-2-(S)-(+)-benzaoate-3-phophosphoramidite glycerol, is elongated using a DNA synthesizer. The reaction can be run, for example, using multiple cycles employing standard coupling conditions and standard cleavage and deprotection conditions to yield the desired PGP polymer. One skilled in the art will also understand that “3” or “5” linking groups, such as amino groups, can be incorporated in the PGP polymer by using a 3′-amino solid support or a 5′-amino phosphoramidite.

### Conjugate Vaccines

Vaccine preparations should be immunogenic, that is, they should be able to induce an immune response. It is not always possible, however, to stimulate antibody formation in a subject merely by injecting a foreign agent. While certain agents can innately trigger the immune response, and may be administered in vaccines without modification, other important agents are not immunogenic and must be converted into immunogenic molecules or constructs before they can induce the immune response.

The immune response is a complex series of reactions that can generally be described as follows: (1) the antigen enters the body and encounters antigen-presenting cells which process the antigen and retain fragments of the antigen on their surfaces; (2) the antigen fragment retained on the antigen presenting cells are recognized by T cells that provide help to B cells; and (3) the B cells are stimulated to proliferate and divide into antibody forming cells that secrete antibody against the antigen.

Most antigens only elicit antibodies with assistance from T cells and, hence, are known as T-dependent (TD). These antigens, such as proteins, can be processed by antigen presenting cells and thus activate T cells in the process described above. Examples of such T-dependent antigens include tetanus and diphtheria toxoids.

Some antigens, such as polysaccharides, cannot be properly processed by antigen presenting cells and are not recognized by T cells. These antigens do not require T cell assistance to elicit antibody formation but can activate B cells directly and, hence, are known as T-independent antigens (TI). PGP is a T-independent antigen.

T-dependent antigens vary from T-independent antigens in a number of ways. Most notably, the antigens vary in their need for adjuvants that will nonspecifically enhance the immune response. The vast majority of soluble T-dependent antigens elicit only low level antibody responses unless they are administered with an adjuvant. Insolubilization of TD antigens into an aggregated form can also enhance their immunogenicity, even in the absence of adjuvants. In contrast, T-independent antigens can stimulate antibody responses when administered in the absence of an adjuvant, but the response is generally of lower magnitude and shorter duration.

Four other differences between T-independent and T-dependent antigens are: (1) T-dependent antigens can prime an immune response so that a memory response can be elicited upon secondary challenge with the same antigen, while T-independent antigens are unable to prime the immune system for secondary responsiveness; (2) the affinity of the antibody for antigen increases with time after immunization with T-dependent but not T-independent antigens; (3) T-dependent antigens stimulate an immature or neonatal immune system more effectively than T-independent antigens; and (4) T-dependent antigens usually stimulate IgM, IgG1, IgG2a, IgG2b, and IgE antibodies, while T-independent antigens mainly stimulate IgM and IgG3 antibodies.

One approach to enhance the immune response to T-independent antigens involves conjugating them to one or more T-dependent antigens. Recruitment of T cell help in this way has been shown to provide enhanced immunity. Conjugate vaccines comprising T cell-independent antigens covalently linked to immunogenic “carrier” proteins capable of recruiting CD4+ T cell help have been shown to elicit high-titer protective IgG responses and to generate immunologic memory against the T-independent antigen.

The carrier protein may be any viral, bacterial, parasitic, animal, or fungal protein/toxoid capable of activating and recruiting T-cell help. Exemplary carrier proteins include, but are not limited to, Tetanus toxoid (TT), diphtheria toxoid (DT), a genetically detoxified diphtheria toxin (e.g., CRM197) (DT), pertussis toxoid (PT), recombinant exoprotein A (rEPA), recombinant staphylococcal enterotoxin Cl (rSEC), cholera toxin B (CTB), meningococcal P64k protein, recombinant PorB (meningococcal porin), Moraxella catarrhalis outer membrane proteins CD and UspA, recombinant Bacillus anthracis protective antigen, recombinant pneumolysin Ply, autolysin (Aly), Klebsiella pneumonia OmpA protein, flagella, nontypeable Haemophilus influenza outer membrane protein P6, recombinant Klebsiella pneumonia outer membrane 40-kDa protein (P40), and outer membrane protein complex (OMPC) from N. meningitidis. In addition, a series of pan HLA-DR-binding peptides (Pan DR helper T cell epitopes; PADRE) have also been used as carrier proteins, and were found to be approximately 1,000 times more powerful than natural T cell epitopes. Historically, conjugate vaccines with a molar TI:TD ratio of about 1:1 to 50:1 have been found to be highly immunogenic and to serve as effective vaccine antigens.

Accordingly, in one aspect of the invention, the PGP is covalently linked to a protein, a toxoid, a peptide, a T-cell or B-cell adjuvant, a lipoprotein, a heat shock protein, a T-cell superantigen, and/or bacterial outer-membrane protein. In another aspect, the PGP is covalently linked to albumin, tetanus toxoid (TT), diptheria toxoid (DT), CRM197, rEPA, pertussis toixoid (PT), KLH, outer membrane protein complex (OMPC), and/or Pan DR helper T cell epitopes (PADRE). In one aspect of the invention, the molar ratio of PGP to carrier protein is about 1:1 to 50:1. In another aspect of the invention, the molar ratio of PGP to carrier protein is about 1:1, 5:1, 10:1, 15:1, 20:1, 25:1, 30:1, 35:1, 40:1, 45:1, or 50:1.

Methods for conjugating T-cell dependent antigens to T-cell independent antigens are known in the art. The carrier compound may be directly linked to the T-cell independent antigen, or may be connected through a linker. In general, at least one moiety must be “activated” to render it capable of covalently bonding to the other molecule. Many conjugation methods are known in the art. (See, e.g., Dick W. E. et al., Contrib. Microbiol. Immunol., 10:48-114 (1989); Hermanson G. T., Bioconjugate Techniques, 2^{nd} Ed. (2008); and U.S. Pat. Nos. 5,849,301 and 5,955,079.) Conjugates can be prepared by direct reductive amination methods, for example, as described in U.S. Pat. Nos. 4,365,170 and 4,673,547. The conjugation method may alternatively rely on activation of hydroxyl groups of the T-cell independent antigen with 1-cyano-4-dimethylamino pyridinium tetrafluoroborate (CDAP) to form a cyanate ester. The activated antigen may then be coupled directly or indirectly (via a linker group) to an amino group on the carrier protein. For example, the cyanate ester can be coupled with hexane diamine or adipic acid dihydrazide (ADH or AH) and then conjugated to the carrier protein using carbodiimide (e.g., EDAC or EDC) chemistry via a carboxyl group on the carrier protein. Such conjugates are described in WO 93/15760, WO 95/08348, and WO 96/29094.

In general, the following types of chemical groups on a protein carrier can be used for coupling/conjugation: (1) carboxyl (e.g., via aspartic acid or glutamic acid), which may be conjugated to natural or derivatized amino groups on T-independent moieties using carbodiimide chemistry; (2) amino group (e.g., via lysine), which may be conjugated to natural or derivatized carboxyl groups on T-independent moieties using carbodiimide chemistry; (3) sulphydryl (e.g., via cysteine); (4) hydroxyl group (e.g., via tyrosine); (5) imidazol group (e.g., via histidine); (6) guanidol group (e.g., via arginine); and (7) indolyl group (e.g. via tryptophan). In a T-cell independent antigen, the following groups can be used for coupling: OH, COOH, or NH₂. Aldehyde groups can be generated by different treatments known in the art including periodate, acid hydrolysis, hydrogen peroxide, etc.

As stated above, the conjugation between the TI moiety and the TD moiety may proceed either indirectly or directly. In certain instances, the process of combining the II moiety and TD moiety may lead to undesirable side effects. For example, direct coupling can place the TI and TD moieties in very close proximity to one another and encourage the formation of excessive crosslinks between the two moieties. Under the extreme of such conditions, the resulting conjugate product can become undesirably thick (e.g., in a gelled state).

Over-crosslinking also can result in decreased immunogenicity of the resulting conjugate product. In addition, crosslinking can result in the introduction of foreign epitopes into the conjugate or can otherwise be detrimental to production of a useful vaccine. The introduction of excessive crosslinks exacerbates this problem.

Control of crosslinking between the TI and TD moieties can be controlled by the number of active groups on each, their concentration, the pH of the reaction, buffer composition, temperature, the use of linkers, and other means well-known to those skilled in the art. (See, e.g., U.S. Publication No. 2005/0169941.)

For example, a linker may be provided between the TI and TD moieties in order to control the degree of crosslinking. The linker helps maintain physical separation between the molecules, and it can be used to limit the number of undesirable crosslinks. As an additional advantage, linkers also can be used to control the structure of the resultant conjugate. If a conjugate does not have the correct structure, problems can result that can adversely affect immunogenicity. The speed of coupling, either too fast or too slow, also can affect the overall yield, structure, and immunogenicity of the resulting conjugate product. (See, e.g., Schneerson et al., Journal of Experimental Medicine, 152: 361 (1980).)

With these considerations in mind, the inventors have developed conjugation methods that produce highly immunogenic PGP conjugates, as set forth in Examples 3 and 7. In one aspect, the PGP molecule is attached to a TD moiety through a linker. In yet another aspect, the linker is attached to the PGP before coupling to the TD moiety. Accordingly, in one aspect, the invention relates to thio-ether coupling of PGP to T-cell dependent antigens. In another aspect, the invention relates to carboxyl coupling of PGP to T-cell dependent antigens. In yet another aspect, the invention relates to the use of oxime chemistry for conjugating PGP to a T-cell dependent antigen.

### Immunogenic Compositions

The invention also relates to immunogenic compositions comprising the PGP antigens of the invention. The compositions of the invention are useful for many in vivo and in vitro purposes. For example, the compositions of the invention are useful for producing an antibody response, for example, as a vaccine for active immunization of humans and animals to prevent staphylococci infection and infections caused by other species of bacteria that contain PGP; as a vaccine for immunization of humans or animals to produce anti-PGP antibodies that can be administered to other humans or animals to prevent or treat infections by Gram-positive bacteria expressing the PGP moiety; as an antigen to screen for important biological agents such as monoclonal antibodies capable of preventing infection by such bacteria, libraries of genes involved in making antibodies, or peptide mimetics; as a diagnostic reagent for staphylococci infections and infections caused by other species of bacteria that contain PGP; and as a diagnostic reagent for determining the immunologic status of humans or animals in regard to their susceptibility to staphylococci infections and infections caused by other species of bacteria that contain PGP.

The compositions of the invention may be administered to any subject capable of eliciting an immune response to an antigen but are especially adapted to induce active immunization against systemic infection caused by staphylococci in a subject capable of producing an immune response and at risk of developing a staphylococcal infection. A “subject capable of producing an immune response and at risk of developing a staphylococcal infection” is a mammal possessing an immune system that is at risk of being exposed to environmental staphylococci or other Gram-positive bacteria that express a PGP moiety. For instance, hospitalized patients are at risk of developing infection as a result of exposure to the bacteria in the hospital environment. High risk populations for developing infection by S. aureus include, for example, renal disease patients on dialysis, and individuals undergoing high risk surgery. High risk populations for developing infection by S. epidermidis include, for example, patients with indwelling medical devices. In some embodiments, the subject is a subject that has received a medical device implant and, in other embodiments, the subject is one that has not received a medical device implant.

The compositions of the invention are administered to the subject in an effective amount for inducing an antibody response. An “effective amount for inducing an antibody response” as used herein is an amount of PGP which is sufficient to (1) assist the subject in producing its own immune protection by, for example, inducing the production of anti-PGP antibodies in the subject, inducing the production of memory cells, and possibly inducing a cytotoxic lymphocyte reaction, etc. and/or (2) prevent infection from occurring in a subject which is exposed to a Gram-positive bacteria that expresses a PGP moiety. One of ordinary skill in the art can assess whether an amount of PGP is sufficient to induce active immunity by routine methods known in the art.

In general, when administered for therapeutic purposes, the formulations of the invention are applied in pharmaceutically acceptable solutions. Such preparations may routinely contain pharmaceutically acceptable concentrations of salt, buffering agents, preservatives, compatible carriers, adjuvants, and/or other therapeutic ingredients. Suitable carrier media for formulating the compositions of the invention include sodium phosphate-buffered saline and other conventional media. Suitable buffering agents include acetic acid and a salt (1-2% WN); citric acid and a salt (1-3% WN); boric acid and a salt (0.5-2.5% WN); and phosphoric acid and a salt (0.8-2% W/V). Suitable preservatives include benzalkonium chloride (0.003-0.03% WN); chlorobutanol (0.3-0.9% WN); parabens (0.01-0.25% WN); and thimerosal (0.004-0.02% W/V). Generally, the compositions of the invention will contain from about 5 to about 100 μg of antigen. In other embodiments, the compositions of the invention will contain about 10-50 μg of antigen

The compositions of the invention may also include an adjuvant. The term “adjuvant” includes any substance which is incorporated into or administered simultaneously with the PGP of the invention to potentiate an immune response in the subject. Adjuvants include, but are not limited to, aluminum compounds (e.g., aluminum hydroxide and aluminum phosphate) and Freund's complete or incomplete adjuvant. Other materials with adjuvant properties include TLR ligands (e.g., the TLR9 agonist CpG-ODN), BCG (attenuated Mycobacterium tuberculosis), calcium phosphate, levamisole, isoprinosine, polyanions (e.g., poly A:U), lentinan, pertussis toxin, lipid A, saponins, QS-21 and peptides (e.g. muramyl dipeptide). Rare earth salts (e.g., lanthanum and cerium) may also be used as adjuvants. The amount of adjuvant can be readily determined by one skilled in the art without undue experimentation.

The present invention provides pharmaceutical compositions for medical use, which comprise PGP of the invention together with one or more pharmaceutically acceptable carriers and optionally other therapeutic ingredients. The term “pharmaceutically-acceptable carrier” as used herein, and described more fully below, means one or more compatible solid or liquid filler, dilutant, or encapsulating substances that are suitable for administration to a human or other animal.

Compositions suitable for parenteral administration conveniently comprise a sterile aqueous preparation of the PGP, which can be isotonic with the blood of the recipient. Among the acceptable vehicles and solvents that may be employed are water, Ringer's solution, and isotonic sodium chloride solution. In addition, sterile fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for subcutaneous, intramuscular, intraperitoneal, intravenous, etc. administrations may be found in Remington's Pharmaceutical Sciences, Mack Publishing Company, Easton, Pa.

The preparations of the invention are administered in effective amounts. An effective amount, as discussed above, is that amount of PGP antigen that will alone, or together with further doses, induce active immunity. It is believed that dosage ranges of 1 nanogram/kilogram to 100 milligrams/kilogram, depending upon the mode of administration, will be effective. In one embodiment, the dosage range is 500 nanograms to 500 micrograms/kilogram. In another embodiment, the dosage range is 1 microgram to 100 micrograms/kilograms. The absolute amount will depend upon a variety of factors including whether the administration is performed on a high risk subject not yet infected with the bacteria or on a subject already having an infection, the concurrent treatment, the number of doses, and the individual patient parameters including age, physical condition, size and weight. These are factors well known to those of ordinary skill in the art and can be addressed with no more than routine experimentation. Generally, a maximum dose should be used that is the highest safe dose according to sound medical judgment.

Multiple doses of the pharmaceutical compositions of the invention are contemplated. Generally, multiple-dose immunization schemes involve the administration of a high dose of an antigen followed by subsequent lower doses of antigen after a waiting period of several weeks. Further doses may be administered as well. Any regimen that results in an immune response to bacterial infection and/or subsequent protection from infection may be used. Desired time intervals for delivery of multiple doses can be determined by one of ordinary skill in the art employing no more than routine experimentation.

A variety of administration routes are available. The particular mode selected will depend upon the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. In one embodiment, the mode of administration is parenteral. The term “parenteral” includes subcutaneous injections, intravenous, intramuscular, intraperitoneal, intrasternal injection, or infusion techniques. Other routes include but are not limited to oral, nasal, dermal, sublingual, and local.

The compositions may conveniently be presented in unit dosage form and may be prepared by any of the methods well known in the art of pharmacy. Other delivery systems can include time-release, delayed release, or sustained release delivery systems. Many types of release delivery systems are available and known to those of ordinary skill in the art. They include polymer based systems such as polylactic and polyglycolic acid, polyanhydrides, and polycaprolactone; nonpolymer systems that are lipids including sterols such as cholesterol, cholesterol esters and fatty acids or neutral fats such as mono-, di- and triglycerides; hydrogel release systems; silastic systems; peptide based systems; wax coatings; compressed tablets using conventional binders and excipients; partially fused implants; and the like.

The PGP antigens of the invention may be delivered in conjunction with other active agents. For example, PGP may be delivered with one or more antibiotics, one or more other antigens, such as bacterial antigens, and/or one or more anti-bacterial antibodies. Such agents are known to those skilled in the art. The PGP and other active agent may be combined in the same composition, or may be administered in separate compositions. If administered in separate compositions, the PGP composition may be administered simultaneously with the other composition, or may be administered sequentially with the other composition.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLES

### Example 1

### Synthesis of PGP

Because the repeating unit in PGP is a phosphoglycerol, it was postulated that this repeating polymer could be synthesized by preparing an appropriately substituted phosphoramidite monomer and elongating it stepwise on a DNA synthesizer. To that end a phosphoramidite synthon was required. Phosphoramidite was designed wherein the 2-OH was protected as its benzoyl ester that would be deprotected with ammonium hydroxide during the cleavage of the polymer from the resin during its solid phase synthesis. The synthesis scheme for producing the 2-OH protected phosphoramidite is shown in FIG. 2.

Briefly, to synthesize phosphoramidite, orthogonally cleavable protecting groups were prepared on the alcohols of glycerol starting from chirally pure acetonide while maintaining the desired chirality at C-2. The scheme in FIG. 2 presents the successful route developed following examination of a wide variety of protection schemes. The alcohol group on compound 1 was protected as its levulinate followed by deprotection of the acetonide with aqueous acetic acid. The primary alcohol of levulinate 3 was protected as its dimethyoxytrityl as required for the DNA synthesizer. The 2-OH was protected as its benzoyl ester and the levulinate was cleaved with pyridine-buffered hydrazine. Alcohol 6 was combined with phosphine 7 to obtain desired phosphoramidites 8 under standard phosphitylation conditions with chloride. The yields of all steps were >70%.

Further details regarding the synthesis scheme shown in FIG. 2 are set forth below:

### (S)-(+)-1,2-Isopropylidene-glycer-3-yl levulinate 2

To a solution of (S)-(+)-1,2-Isopropylideneglycerol 1 (15.00 g; 0.114 mol; SigmaAldrich, St. Louis, Mo.) in dichloromethane (200 mL) was added levulinic acid (13.2 g; 1.114 mol; SigmaAldrich) and DMAP (2.78 g; 0.023 mol; SigmaAldrich). To the stirred solution was added dropwise a solution of dicyclohexylcarbodiimide (DCC; 26.15 g; 0.114 mol; SigmaAldrich). The reaction was stirred for 4 hours and the precipitated DCU was removed by filtration. The reaction was shown to be complete by TLC (hexanes/ethyl acetate (1/1); PMA development). The reaction mixture was washed with saturated sodium bicarbonate solution and brine, dried over anhydrous sodium sulfate, filtered and concentrated to yield 2 (25.7 g; 98.2% yield) as a colorless oil.

### (S)-(+)-Glycer-3-yl Levulinate 3

To a solution AcOH/H₂O (3/1; 120 mL) was added acetonide 2 (25.0 g). The reaction mixture was heated in a 50° C. oil bath for 16 h. The reaction mixture was concentrated to a thick oil and coevaporated twice from xylenes. A fraction of the crude (16 g) was purified by flash chromatography on silica gel initially using hexane/ethyl acetate (1/2) as eluant followed by 100% ethyl acetate. The fractions containing product were pooled and concentrated to yield 3 (10.2 g).

### (S)-(+)-1-DMT-glycer-3-yl Levulinate 4

To a solution diol 3 (7.35 g; 38.6 mmol) in anhydrous pyridine (200 mL) was added dropwise a solution of dimethoxytrityl chloride (13.1 g; 38.6 mmol; SigmaAldrich). Following stirring for 2 h at room temperature the reaction was shown to be complete by TLC (hexanes/ethyl acetate (1/2); UV and acid development). The solvent was removed on the rotary evaporator, dissolved in dichloromethane and washed with saturated sodium bicarbonate and brine. The organic phase was dried over anhydrous sodium sulfate, filtered and concentrated to give a pale yellow. The crude product was coevaporated from xylenes (2×). The product was purified by flash chromatography.

### (S)-(+)-1-DMT-2-benzoate-glycer-3-yl Levulinate 5

To a solution of 4 (11.60 g; 23.6 mmol) in anhydrous pyridine was added a solution of benzoyl chloride (4.15 g; 2.95 mmol) in anhydrous pyridine (6 mL). The reaction mixture was stirred at room temperature for 1.5 h. TLC (hexanes/ethyl acetate (1/1)) indicated excellent conversion to product. The solvent was removed on the rotary evaporator, dissolved in dichloromethane and washed with saturated sodium bicarbonate and brine. The organic phase was dried over anhydrous sodium sulfate, filtered and concentrated to give a pale yellow. The crude product was coevaporated from xylenes (2×). The product was purified by flash chromatography to yield 6.5 g 5 as a viscous oil.

### (S)-(+)-1-DMT-2-benzoate-glycerol 6

A solution of 1 M hydrazine hydrate (1.21 mL; 2.01 mmol) in pyridine/AcOH (3/2; 20 mL). To a solution of 5 (6.00 g; 10.1 mmol) in pyridine was added the hydrazine/pyridine/AcOH solution and the reaction mixture was stirred at room temperature for 1.5 h. TLC indicated complete consumption of starting material 5 (the Rf of starting material 5 is slightly lower than the product). The solvent was removed on the rotary evaporator and the residue was dissolved in dichloromethane, washed with saturated sodium bicarbonate (2×), 5% LiCl and brine. The organic phase was dried over anhydrous sodium sulfate, filtered and concentrated to give a viscous oil. The product was purified by flash chromatography on a silica gel column pre-equilibrated sequentially with 5% triethylamine in hexanes, hexanes and hexanes/ethyl acetate (3/1). The crude product was applied to column and initially eluted with hexanes/ethyl acetate (3/1) to remove high Rf impurities and then eluted with hexanes/ethyl acetate (2/1). Fractions containing product were pooled and concentrated to yield 6 (4.0 g) as a viscous oil.

### Amidite 8

Under anhydrous conditions to a solution of alcohol 6 (3.80 g; 7.62 mmol) and N,N-diisopropylethylamine (1.1 equivalents) in anhydrous DCM (30 ml) was added 3-((chloro(diisopropylamino) phosphino)oxy)propanenitrile (2.25 g; 2.0 mL; Chemgenes, Wilmington, Mass.) dropwise by syringe. The reaction mixture was stirred at room temperature for 30 min and the reaction was shown to be complete by TLC (hexanes/ethyl acetate/TEA (67/33/2); UV and acid development). MeOH (1 mL) was added to the reaction mixture and the reaction mixture was stirred for 5 min then concentrated to dryness on the rotary evaporator. The residue was dissolved in dichloromethane and washed with saturated sodium bicarbonate (2×) and brine, dried over anhydrous sodium sulfate, filtered and concentrated to give a colorless oil. The product was purified by flash chromatography on a silica gel column pre-equilibrated sequentially with 5% triethylamine in hexanes, hexanes, and hexanes/ethyl acetate (2/1). The crude product was applied to column and initially eluted with hexanes/ethyl acetate (3/1) to remove high Rf impurities and then eluted with hexanes/ethyl acetate (2/1). Fractions containing product were pooled and concentrated to yield 8 (4.2 g; 78.9%) as a viscous oil.

### Phosphate-PGP-12-mer-hexylamino synthesis

The synthesis of the PGP incorporating an amino group at the one terminus and phosphate at the other was accomplished using standard solid support DNA synthetic methods at Allele Biotechnology (www.allelebiotech.com) using GeneMachine's PolyPlex Oligo synthesizer and 3′-Amino-Modifier C7 CPG support (Allele Biotechnology). In each cycle, amidite 8 (35-40 equivalents) was used. Following 12 cycles, the polymer was deprotected and cleaved using standard ammonium hydroxide cleavage conditions, i.e., aqueous ammonium hydroxide, 55° C. The product isolated as a viscious oil and was lyophilized. The lyophilized product was resuspended in water and desalted using a 3K dialysis cassette for conjugation.

### Example 2

### Recognition of PGP by Pagibaximab

ELISA plates (96-well) were coated overnight with 4 mg/ml of Pagibaximab (anti-LTA), Zantibody (anti-peptidoglycan), or Synagis (anti-RSV), all of which are chimeric IgG1 antibodies. PGP was prepared as described above in Example 1, and LTA was extracted from S. aureus. Both molecules were biotinylated and then added to the ELISA plates, at the concentrations indicated in Table 1, for one hour. The plates were then developed with horseradish peroxidase for 30 minutes. Table 1 presents the O.D. readings obtained from each well. These experiments show PGP was recognized by a monoclonal antibody (mAb) specific for LTA (Pagibaximab), suggesting that PGP is a potential vaccine target for Gram-positive bacteria containing PGP.

TABLE 1
The anti-LTA antibody Pagibaximab exhibits equivalent
reactivity with lipoteichoic acid (LTA) and
(poly)glycerolphosphate (PGP).

| | | | Anti- | | | |
|---|---|---|---|---|---|---|
| | Pagibaximab | | peptidoglycan | | Synagis | |
| μg/ml | LTA | PGP | LTA | PGP | LTA | PGP |
| 3 | 1.47 | 1.91 | 0.05 | 0.03 | <0.02 | <0.02 |
| 1 | 1.25 | 1.42 | <0.02 | <0.02 | <0.02 | <0.02 |
| 0.33 | 1.23 | 1.09 | <0.02 | <0.02 | <0.02 | <0.02 |
| 0.1 | 1.16 | 0.89 | <0.02 | <0.02 | <0.02 | <0.02 |
| 0.037 | 1.05 | 0.67 | <0.02 | <0.02 | <0.02 | <0.02 |
| 0.012 | 0.88 | 0.51 | <0.02 | <0.02 | <0.02 | <0.02 |
| 0.004 | 0.67 | 0.34 | <0.02 | <0.02 | <0.02 | <0.02 |
| 0 | <0.02 | <0.02 | <0.02 | <0.02 | <0.02 | <0.02 |
| (PBS) | | | | | | |

In a separate experiment, ELISA plates (96-well) were coated overnight with 4 mg/ml of Pagibaximab. LTA was added to biotin-PTP or biotin-LTA for 2 hours, and the mixture was then added to the Pagibaximab-coated wells. After 60 minutes, the plates were washed and horseradish peroxidase was added for an additional 30 minutes. Table 2 shows the percent inhibition of binding of LTA or PGP to the Pagibaximab-coated wells in the presence of excess LTA. These results demonstrate that LTA can inhibit PGP binding to Pagibaximab.

TABLE 2
LTA blocks the binding of biotin-PGP and biotin-LTA to Pagibaximab-
coated ELISA plates.

| Concentration LTA | % Inhibition Binding of | % Inhibition Binding of |
|---|---|---|
| (μg/ml) | Biotin-LTA | Biotin-PGP |
| 50 | 95 | 91 |
| 40 | 92 | 74 |
| 30 | 77 | 31 |
| 20 | 64 | 26 |

### Example 3

### Conjugation of PGP to T-Cell Dependent Carrier

Approximately 1.5 mg of crude synthetic PGP 10-mer containing an amino linker was solubilzed in 300 μl of water and dialyzed against water using a dialysis cassette with a 2 kDa cutoff (Pierce). The dialyzed material was made pH 7.3 and labeled using an excess of GMBS. After 1 hour, free reagent was removed by an overnight dialysis. Six (6) mg of tetantus toxoid (obtained from the Serum Institute of India) was made pH 8 and labeled with an approximately 50 fold molar excess of SPDP. After an overnight reaction, the solution was adjusted to pH 6.8 and made about 25 mM DTT. After about 30 min, the solution was desalted on a 1×15 cm G25 column equilibrated with PBS+5 mM EDTA, pH 6.8. The protein fractions were concentrated using an Amicon Ultra4, 30 kDa cutoff device to a final concentration of about 64 mg. The protein was then combined with the maleimide-PGP at an approximate ratio of 10 PGP/TT. After 4 hr, the solution was made about 10 mM in N-ethylmaleimide and the pH adjusted to 8. Free reagent was removed using the Amicon device, with repeated washes of 0.1 M sodium borate, pH 9. The protein concentration was determined from its absorbance, and the solution was assayed for phosphate and the PGP content determined. The final conjugate was found to have approximately 10 mole PGP/mole TT.

### Example 4

### Immunization of Mice with PGP-Conjugate Vaccine

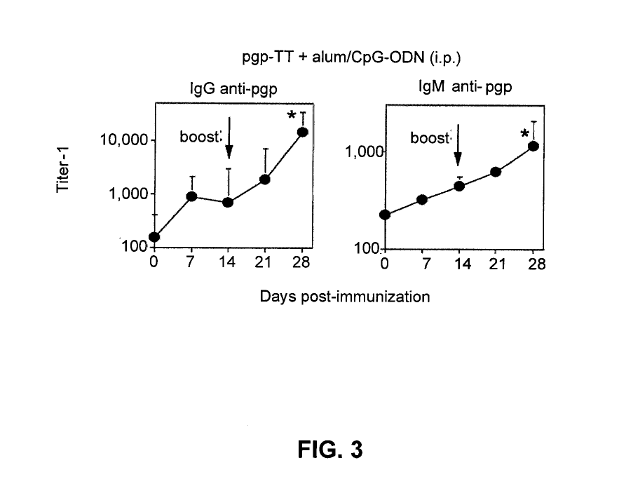

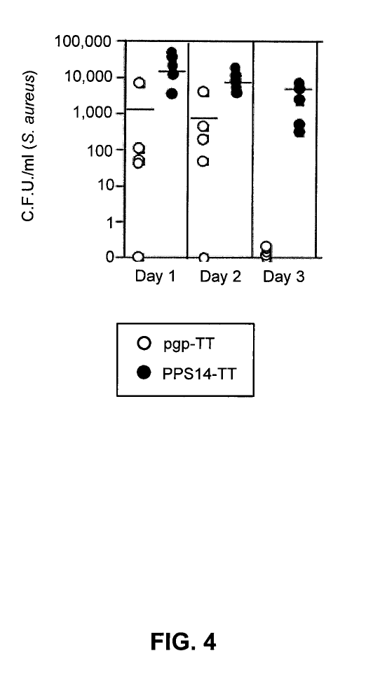

A synthetic PGP molecule containing 10 glycerol phosphate monomers (PGP₁₀) was produced using the method described above in Example 1. PGP₁₀ was then covalently attached to tetanus toxoid (TT) at a ratio of about 6 PGP per TT molecule using a hexylamine linker on the PGP, as described above in Example 3. Mice were immunized (7 mice per group) with 1 μg of conjugate in 13 μg alum and 75 μg CpG-ODN. On day 14, the mice were boosted with an additional 1 μg of the conjugate vaccine. Immunization and boosting of BALB/c mice intraperitoneally (i.p.) with PGP-TT in the presence of the adjuvanting molecules alum and CpG oligodeoxynucleotides (ODN) elicited high-titers of serum IgG anti-PGP antibody, as measured by a PGP-specific ELISA (FIG. 3). Blood was obtained from the tail vein on days 1, 2, and 3, and colony counts were performed on agar plates. Mice immunized in this fashion rapidly cleared live S. aureus from the blood following i.p. infection, relative to mice immunized in a similar fashion with a conjugate vaccine, comprising pneumococcal capsular polysaccharide type 14 (PPS14) covalently attached to TT (PPS14-TT) (FIG. 4).

These data suggest that a PGP-based conjugate vaccine provide active protection against infections with multiple species of Staphylococci, as well as other Gram-positive bacteria expressing PGP-containing LTA.

### Example 5

### Optimizing the Immunogenicity of PGP-Conjugates as a Function of the Number of Glycerol Phosphate Monomers

The immunogenicity of the PGP-conjugates of the invention may be optimized as a function of the number of glycerol phosphate monomers. To this end, a series of PGP molecules containing, for example, 5, 10, 15, and 20 glycerol phosphate monomers may be synthesized as described above in Example 1. The PGP molecules may be terminated with a C6 amino group for subsequent modification with a linker moiety. The products may be characterized by mass spectral analysis. These PGP molecules may then be covalently linked to GMP vaccine-grade TT as described above in Example 3.

The conjugates may be used to immunize female BALB/c mice (5 weeks of age) (7 mice per group). BALB/c mice have been found to be more sensitive to infection with S. aureus than other strains, and younger mice are more sensitive than older mice. The mice may be injected i.p. with the PGP-TT conjugates (0.2, 1.0, 5.0, or 25 μg/mouse) adsorbed to 13 μg of alum (Allhydrogel 2%) and boosted in a similar fashion on day 14. Titers of PGP-specific IgG may be determined, from serum samples obtained on days 0, 7, 14 (primary), 21 and 28 (secondary) from blood obtained through the tail vein, utilizing an ELISA assay. Briefly, ELISA plates may be coated with avidin followed by addition of biotin-PGP. Plates may then be blocked with PBS+1% BSA. Threefold dilutions of serum samples, starting at a 1/50 serum dilution in PBS+1% BSA may then be added. Alkaline phosphatase-conjugated polyclonal goat anti-mouse IgM, IgG, IgG3, IgG1, IgG2b, or IgG2a Abs may then be added followed by substrate (p-nitrophenyl phosphate, disodium) for color development. Color may be read at an absorbance of 405 nm on a Multiskan Ascent ELISA reader.

A standard curve may be generated using a PGP-specific murine IgG1 monoclonal antibody (clone M110) in order to directly compare data from multiple experiments. Serum may be further tested for opsonophagocytic activity in vitro using both community acquired methicillin-resistant (MRSA) NRS123 S. aureus (USA400), capsule type 5 methicillin-sensitive (MSSA) S. aureus (ATCC 49521), and S. epidermidis (strain Hay). These strains have known clinical relevance. S. aureus is more virulent than S. epidermidis, and so is more suited for use in an in vivo infection model, since the latter requires extremely high doses for infectivity. However, protection in vivo against S. epidermidis can be strongly implied through the in vitro opsonophagoctyosis assay alone, especially if these parameters correlate using S. aureus. Serum antigen-specific IgG titers and opsonophagocytosis are expected to correlate well with host protection in vivo.

The S. aureus and S. epidermidis may be grown to mid-log phase using standard growth protocols. Bacterial numbers may be determined by colony counts on blood agar plates. S. aureus may be injected i.v. at varying non-lethal but infective doses (5×10⁶, 1×10⁷, and 2×10⁷ CFU/mouse). Blood for bacterial colony counts may be obtained on days 1, 2, and 3, and colony counts from spleen, liver, and kidney may be determined on day 7.

The opsonophagocytosis assay may be performed as described previously. (Romero-Steiner S. et al., Clin Diagn Lab Immunol., 4:415-422 (1997).) Briefly, sera may be tested for opsonophagoctyosis activity (titers) against S. aureus and S. epidermidis using HL-60 cells (human promyelocytic leukemia), which have been differentiated to neutrophils using N,N-dimethylformamide (4×10⁵ cells in a 40 μL volume). An effector [neutrophils]/target [bacteria] ratio of 400/1 may be used. Bacterial colony counts in HL-60 cell cultures in the presence or absence of immune sera may be scored to calculate titers using an anti-LTA mAb (Pagibaximab) as a positive control. Opsonophagocytic titers are the reciprocal of the serum dilution showing >50% killing compared with growth in control wells. In other words, the serum dilution which results in killing over 50% of the plated bacteria relative to serum from unimmunized mice is used as the “reciprocal serum dilution.” Thus, if a 1/100 serum dilution kills >50% bacteria, whereas a more dilute serum does not, then the number used is 100 (i.e., the reciprocal of 1/100).

The lowest dose of each conjugate that generates the highest serum titers of PGP-specific IgG and/or opsonophagocytic activity in vitro, may be chosen to directly compare the ability of the conjugates to confer host protection against i.v. challenge with live MRSA and MSSA S. aureus, as reflected by the level of bacteremia during the first 3 days, and colony counts of S. aureus obtained from spleen, liver, and kidney on day 7. Three non-lethal doses of bacteria (5×10⁶, 1×10⁷, and 2×10⁷ CFU/mouse), may be injected i.v., 2 weeks following secondary immunization, into 7 BALB/c mice per group. Blood samples from several additional mice, not infected with bacteria may be used as a negative control. Infected mice, which are either unimmunized or immunized with an irrelevant pneumococcal vaccine, may be used as a positive control.

Higher polymer length is expected to correlate with higher serum titers of PGP-specific IgG. Higher serum titers are likely to yield higher levels of in vitro opsonophagocytic activity and better host protection using live bacteria.

### Example 6

### Optimizing the Immunogenicity of PGP-Conjugates as a Function of the Ratio of PGP:Carrier Protein

The immunogenicity of the PGP-conjugates of the invention may be measured as a function of the ratio of PGP to carrier protein. To this end, a series of PGP-TT conjugates may be synthesized using the PGP of optimal polymer length determined above in Example 5 and the conjugation protocol set forth in Example 3, with PGP:TT ratios of approximately 10, 20, and 30. This may be accomplished by varying the molar ratio of N-[γ-maleimidobutyryloxy]succinimide ester (GMBS) to the TT protein to increase the number of reactive sites. Immunizations and analyses of the protective PGP-specific IgG response may be performed as discussed above in Example 5.

Higher PGP:TT molar ratio are expected to correlate with higher serum titers of PGP-specific IgG. Higher serum titers are likely to yield higher levels of in vitro opsonophagocytic activity and better host protection using live bacteria.

### Example 7

### Alternative Conjugation Chemistries

PGP may be synthesized with an aldehyde linker and with a carboxyl linker in addition to the hexylamine described above in Example 3. TT may be functionalized with hydrazides or amino-oxy groups. (See, e.g., WO/2005/072778 and Lopez-Acosta et al., Vaccine, 24:716 (2006).) The PGP and TT may be coupled using one of the chemistries set forth below.

Thio-Ether Coupling to Protein Amines

PGP-NH₂ may be solublized at 10 mg/ml in 0.1 M HEPES, 5 mM EDTA pH 8. A 2-fold molar excess of Sulfo-LC-SPDP may be added as a solid, while stirring. After 1 h, the pH may be reduced to pH 5 and the solution made 25 mM DTT. After 30 min, the solution may be desalted on a G10 desalting column equilibrated with 10 mM sodium acetate, 5 mM EDTA, pH 5. The void volume may be pooled and assayed for thiols using a DTNB assay (Ellman G. L. 1959. Arch Biochem Biophys 82:70-77) and for phosphate. Degassed buffers may be used, and the thiolated-PGP kept under argon. The protein may be solublized at 10 mg/ml in 0.1 M HEPES, pH 8 and bromoacetylated to varying levels using NHS bromoacetate at 0-50 mole/mole protein. After 2 hrs at 4° C., each may be desalted by repeated washes using an Amicon Ultra 15 device (30 kDa and 10 kDa cutoff for TT and CRM197 respectively) into the same buffer. Residual amines may be assayed using TNBS (Vidal J. et al., J Immunol Methods, 86:155-156 (1986)) and the extent of derivatization determined from the decrease in free amines from the native protein. The final concentration of protein may be brought to 10 mg/ml and the solution gently degassed with argon. The PGH-SH and bromoacetylated protein may be combined at a 1.5:1 molar excess of PGP over bromoacetyl groups, the pH adjusted to 8 and the reaction mixture stirred under argon at 4° C. Conjugation kinetics may be determined by periodic sampling, quenching the aliquot with mercaptoethanol, and evaluating by SDS PAGE. Remaining active groups may be quenched with mercaptoethanol and the unconjugated PGP removed by size exclusion chromatography on an S100HR column, equilibrated with HEPES. A control conjugate, without PGP, may be made by incubating the bromoacetylated protein with mercaptoethanol.

Coupling to Protein Carboxyls

PGP-CO₂H may be coupled as follows. A hydrazide-protein (Hz-protein) may be prepared by combining TT at 5 mg/ml in 0.1 M MES buffer, pH 5 plus 0.25M in adipic dihydrazide (ADH). Five mg/ml EDC may be added and the pH maintained at 5.5 for 2 hrs. The solutions may be quenched by the addition of sodium acetate, pH 5.5, and then desalted on a G25 column equilibrated with MES buffer and concentrated to 10 mg/ml using an Amicon Ultra 15 device. The extent of derivatization may be determined using TNBS. PGP-CO₂H may be added to the protein-hydrazide solution at a molar ratio of 50:1 and the solution made 5 mM in carbodiimide. After an overnight incubation, the pH may be raised to 8 and the unconjugated PGP removed by size exclusion chromatography.

Alternative Chemistry for Coupling to Protein Amines

PGP-Ald may be coupled using oxime chemistry. Amino-cm protein (AO-protein) may be prepared as described previously. (Lees A. et al., Vaccine, 24:716-729 (2006).) In brief, the protein may be bromoacetylated as described above and then reacted with a 2-fold excess of thiol-animooxy reagent, followed by desalting into a pH 5 acetate buffer and concentrated to 20 mg/ml. The PGP-Ald at 10 mg/ml in 0.1 M sodium acetate+5 mM EDTA, pH 5 may be combined with the AO-protein at a molar ratio of 1.1:1 and made 5 mM sodium cyanoborohydride. After 4 hrs the reaction solution may be made 5 mM acetoaldehyde and unconjugated PGP removed by size exclusion chromatography

In all cases, the extent of functionalization may be determined from the difference with native protein. Hydrazides or aminooxy groups may be determined using TNBS and absorbance at 550 nm with either ADH or aminooy acetate as the standard. Protein concentration may be determined from the absorbance at 280 nm and the extinction coefficient. PGP concentration may be determined using a phosphate assay. Moles of PGP may be calculated from moles phosphate/#repeat groups per PGP, and the loading determined from moles PGP/mole protein. Conjugation may be assessed using a Western blot with anti-LTA mAb as the detection antibody, and may be further confirmed using a double ELISA in which anti-TT or anti-CRM197 is used as the capture antibody and anti-LTA mAb as the detection antibody. Since aggregation can affect immunogenicity, conjugates may be analyzed by SEC HPLC, using a Phenomenex BioSep G4000 column. Unconjugated PGP may be removed by size exclusion chromatography, since previous studies show that high doses of unconjugated polysaccharide in a conjugate preparation can inhibit the PS-specific Ig response to the conjugate itself.

Immunizations and analyses of the protective PGP-specific IgG response may be performed as discussed above in Example 5.

### Example 8

### Optimizing the Immunogenicity of PGP-Conjugates as a Function of the Carrier Protein and Adjuvant

The immunogenicity of the PGP-conjugates of the invention may be measured as a function of the carrier protein and adjuvant used. Both TT and CRM197 are immunogenic protein carriers that have been used for conjugate vaccines currently in clinical use and thus, have been shown to be effective. TT is more potent than CRM197 for activation of CD4+ T cells, which are critical for generating help for the attached target antigen. Although a significant correlation between IgG anti-PS and IgG anti-carrier responses can be observed in response to conjugate vaccines, this is not always the case, since excessive focus of CD4+ T cell help on carrier-specific B cells may diminish this same help for B cells responding to the attached target antigen. Further, the nature of the attached target antigen can influence the peptide specificity of the CD4+ T cell response to the carrier. Finally, diminished anti-PS responses to conjugate vaccines have been observed when the same carrier protein is used for different vaccine types. These observations suggest it may be helpful to test distinct carriers for induction of a protective IgG anti-PGP response.

In addition, although alum is currently the most commonly used adjuvant for human use, producing relatively minimal side effects, it has relatively limited immunostimulatory properties. In this regard, other adjuvants such as TLR ligands, which elicit considerably higher antibody responses than alum, and more protective IgG isotypes (e.g. IgG2a in mice) have been under investigation, despite their potential for more significant side effects. One such TLR ligand, which has shown promise in various clinical trials, is the TLR9 agonist, CpG-ODN. Thus, inclusion of CpG-ODN to alum in the adjuvant formulation will likely generate data that will have potential clinical utility.

To this end, conjugates of PGP linked to CRM197 or to TT may be synthesized using the PGP of optimal polymer length determined above in Example 5, the optimal PGP:carrier ratio determined in Example 6, and the optimal conjugation chemistry determined in Example 7. Mice may be immunized as discussed above in Example 5 with varying doses of conjugate in alum, in the presence or absence of 25 μg of 30 mer CpG-ODN, and sera may be tested for titers of PGP-specific IgG isotypes and opsonophagocytic activity. In addition, immunogenicity of the conjugates containing different carrier proteins may also be measured in C57BL/6 [MHC-II^{b}], C3H (MHC-II^{k}) and A.SW (MHC-II^{s}) mice in addition to BALB/c (MHC-II^{d}) mice. These results may be compared to those from a breeding colony of MHC-II−/− mice that are transgenic for human HLA-DR4.

The level of induction of protective PGP-specific IgG will likely be directly correlated with the relative strengths of the protein carriers for CD4+ T cell activation, and addition of CpG-ODN to alum will likely enhance the protective PGP-specific IgG response over that seen using alum alone.

In addition, PGP-PADRE conjugates may also be tested, since an entirely synthetic conjugate vaccine has potential advantages over conventional conjugate vaccines in regards to reproducibility, safety, and cost-effectiveness. As discussed above, PADRE was found to be approximately 1,000 times more powerful than natural T cell epitopes, and PADRE-peptide constructs in adjuvant were shown to be immunogenic. Linkage of PADRE to Streptococcus pneumoniae capsular polysaccharides (PPS) augmented the in vivo anti-PPS response in mice through recruitment of PADRE-specific CD4+ T cells. Thus, PADRE might represent a more efficient substitute for intact immunogenic carrier proteins in the formulation of a CD4+ T cell-dependent PGP conjugate vaccine.

To this end, a 13 amino acid PADRE peptide (AKXVAAWTLKAAA where X=cyclohexylalanine) with an N-terminal cysteine may be synthesized using standard peptide chemistry. PGP-NH₂ possessing the optimized chain length determined in Example 5 may be bromacetylated with a 2× molar excess of NHS bromoacetate at pH 8.0, and desalted on a G10 desalting column into 50 mM HEPES+5 mM EDTA. Thiol-PADRE peptide may be added at a 1.5:1 molar ratio of PDRE:PGP. After 2 hrs, the conjugate may be purified using a Pepdex size exclusion column (GE Healthcare #17-5176-01). The reaction progress and purification may be monitored using reverse phase HPLC. PADRE concentration may be determined from the peptide's extinction coefficient and the PGP concentration determined by assaying for phosphate.

Several conjugates may be prepared in which the molar ratio of PADRE to PGP is varied to determine optimal immunogenicity. Mice may be immunized as described above in the presence of alum with or without CpG-ODN. Primary and secondary serum titers of IgG anti-PGP may be determined by ELISA, serum opsonic activity may be determined by the opsonophagoctyosis assay (S. aureus and S. epidermidis), and host protection may be determined by infection with S. aureus. The data generated may be directly compared to that obtained using the optimized PGP-protein natural carrier conjugate determined above. Initial comparative studies may utilize BALB/c mice, but may be extended to using mice of additional mouse MHC-II backgrounds, as well as HLA-DR4 transgenic mice, as described above.

An optimized PGP-PADRE conjugate is expected to exhibit a higher protective PGP-specific IgG response (i.e. serum PGP-specific titers, opsonophagoctyosis, and in vivo host protection) than a corresponding PGP-carrier protein conjugate, per weight of PGP used for immunization, due to a higher efficiency of CD4+ T cell recruitment.

What is claimed is:

1. An immunogenic composition comprising poly-glycerolphosphate (PGP) covalently linked to a T-cell dependent antigen.

2. The immunogenic composition of claim 1, wherein the PGP is produced by preparing a substituted phosphoramidite monomer and elongating it stepwise.

3. The immunogenic composition of claim 1, wherein the PGP comprises about 5-20 glycerol phosphate monomers.

4. The immunogenic composition of claim 3, wherein the PGP comprises about 10 glycerol phosphate monomers.

5. The immunogenic composition of claim 1, wherein the T-cell dependent antigen is tetanus toxoid (TT), diptheria toxoid (DT), genetically detoxified diphtheria toxin, pertussis toixoid (PT), recombinant exoprotein A (rEPA), outer membrane protein complex (OMPC), or a Pan DR helper T cell epitope (PADRE) peptide.

6. The immunogenic composition of claim 5, wherein the genetically detoxified diphtheria toxin is CRM197.

7. The immunogenic composition of claim 5, wherein the T-cell dependent antigen is a PADRE peptide.

8. The immunogenic composition of claim 7, wherein the PADRE peptide comprises the sequence AKXVAAWTLKAAA, wherein X is cyclohexylalanine.

9. The immunogenic composition of claim 1, wherein the molar ratio of PGP to the T-cell dependent antigen is about 5:1 to 50:1.

10. The immunogenic composition of claim 9, wherein the molar ratio is 10:1.

11. The immunogenic composition of claim 1, wherein the PGP is directly linked to the T-cell dependent antigen.

12. The immunogenic composition of claim 1, wherein the PGP is linked to the T-cell dependent antigen through a linker.

13. The immunogenic composition of claim 1, wherein the PGP is covalently linked to the T-cell dependent antigen using a thiol group, a thiol-ether group, an acyl-hydrazone group, a hydrazide group, a hydrazine group, a hydrazone group, or an aminooxy group.

14. The immunogenic composition of claim 13, wherein the hydrazone group is a bis-arylhydrazone group.

15. The immunogenic composition of claim 13, wherein the thiol nucleophile group is succinimidyl 6-[3-(2-pyridyldithio)-propionamido] hexanoate (SPDP), or N-succinimidyl-5-acetylthioacetate (SATA).

16. The immunogenic composition of claim 13, wherein the hydrazide nucleophile group is added using E-maleimidocaprioc acid hydrazide-HCl (EMCH), or hydrazine or adipic dihydrazide (ADH) and 1-ethyl-3-dimethylaminopropyl)carbadiimide hydrochloride (EDC), and the arylhydrazine group is added using succinimidyl hydrazinonicotinate acetone hydrazone.

17. A method for treating an infection by a bacteria expressing PGP comprising administering an effective amount of the immunogenic composition of claim 1.

18. The method of claim 17, wherein the bacteria is staphylococci.

19. The method of claim 18, wherein the bacteria is Staphylococcus aureus or Staphylococcus epidermidis.

20. The method of claim 17, wherein the immunogenic composition is administered parenterally.

21. The method of claim 17, wherein the immunogenic composition is administered with another active agent.

22. The method of claim 21, wherein the other active agent is an antibiotic, a bacterial antigen, or an anti-bacterial antibody.

23. A method for vaccinating a subject against a bacteria expressing PGP comprising administering an effective amount of the immunogenic composition of claim 1.

24. The method of claim 23, wherein the bacteria is staphylococci.

25. The method of claim 24, wherein the bacteria is Staphylococcus aureus or Staphylococcus epidermidis.

26. The method of claim 23, wherein the immunogenic composition is administered parenterally.

27. The method of claim 23, wherein the immunogenic composition is administered with another active agent.

28. The method of claim 27, wherein the other active agent is an antibiotic, a bacterial antigen, or an anti-bacterial antibody.

29. A method for generating protective antibodies against a bacteria expressing PGP comprising administering an effective amount of the immunogenic composition of claim 1.

30. The method of claim 29, wherein the bacteria is staphylococci.

31. The method of claim 30, wherein the bacteria is Staphylococcus aureus or Staphylococcus epidermidis.

32. The method of claim 29, wherein the immunogenic composition is administered parenterally.

33. The method of claim 29, wherein the immunogenic composition is administered with another active agent.

34. The method of claim 33, wherein the other active agent is an antibiotic, a bacterial antigen, or an anti-bacterial antibody.

35. A method for synthesizing poly-glycerolphosphate (PGP) comprising preparing a protected and activated phosphoramidite monomer and elongating the monomer stepwise.

36. The method of claim 35, wherein the elongation comprises standard solid phase oligonucleotide synthetic technology.

37. The method of claim 36, wherein the elongation is performed on a DNA synthesizer.

38. The method of claim 35, wherein a linking group is incorporated on the PGP during the elongation.

39. The method of claim 38, wherein the linking group is incorporated by a solid support during the elongation.

40. The method of claim 38, wherein the linking group is an amino group.

41. The method of claim 35, wherein the monomer comprises a linking group or a precursor to a linking group.

42. The method of claim 41, wherein the linking group is an amino group.

43. The method of claim 35, wherein the monomer is a glycerol molecule comprising:
(a) an acid labile protecting group on one terminal hydroxyl group;
(b) a base labile group on the 2-OH; and/or
(c) an activated phosphorus group on the other terminal hydroxyl.

44. The method of claim 43, wherein the glycerol is chirally pure.

45. The method of claim 44, wherein the activated phosphorus group comprises a linking group or a precursor to a linking group.

46. The method of claim 45, wherein the linking group is an amino group.

47. The method of claim 44, wherein the base labile group of (b) is stable to acid deprotection conditions.

48. The method of claim 44, wherein the elongation comprises standard solid phase oligonucleotide synthetic technology using a solid phase support, wherein the base labile group of (b) is removed during the cleavage of the PGP from the solid phase support.

49. The method of claim 35, wherein the monomer is prepared by:
(a) protecting a glycerol molecule with an acid labile protecting group on one terminal hydroxyl group;
(b) protecting a glycerol with a base labile group on the 2-OH; and/or
(c) protecting a glycerol with an activated phosphorus group on the other terminal hydroxyl.

50. The method of claim 49, wherein the glycerol molecule is chirally pure.

51. The method of claim 50, wherein the activated phosphorus group comprises a linking group or a precursor to a linking group.

52. The method of claim 51, wherein the linking group is an amino group.

53. The method of claim 50, wherein the base labile group of (b) is stable to acid deprotection conditions.

54. The method of claim 50, wherein the elongation comprises standard solid phase oligonucleotide synthetic technology using a solid phase support, wherein the base labile group of (b) is removed during the cleavage of the PGP from the solid phase support.

55. The method of claim 50, wherein the glycerol molecule is first protected with the acid labile protecting group and then protected with the base labile group.

56. The method of claim 35, wherein the monomer is prepared by:
(a) preparing a levulinate ester from an isopropylidene glycerol molecule;
(b) removing the isopropylidene protecting group;
(c) protecting the free terminal alcohol with an acid labile group;
(d) protecting the 2-OH group with a base labile group;
(e) deprotecting the levulinate ester to provide a free terminal hydroxy; and
(f) phosphitylating the free terminal alcohol.

57. The method of claim 56, wherein the isopropylidene glycerol molecule is chirally pure.

58. The method of claim 56, wherein the levulinate ester is removed by hydrazine.

59. A synthetic poly-glycerolphosphate (PGP) molecule produced by the method of claim 37.

60. The synthetic PGP of claim 59, wherein the linker group comprises a thiol, amine, aminooxy, aldehyde, hydrazide, hydrazine, maleimide, carboxyl, or haloacyl.

61. A synthetic poly-glycerolphosphate (PGP) molecule comprising a linker group.

62. The synthetic PGP of claim 61, wherein the linker group comprises a thiol, amine, aminooxy, aldehyde, hydrazide, hydrazine, maleimide, carboxyl, or haloacyl.
